(19) United States
(12) Patent Application Publication
(10) Pub. No.: US 2020/0095192 A1
(43) Pub. Date: Mar. 26, 2020

(54) BIODEGRADABLE DIETHANOLAMINE DERIVATIVE CHELATING AGENT AND PREPARATION PROCESS THEREOF

(71) Applicant: PTT Global Chemical Public Company Limited, Chatuchak, Bangkok (TH)

(72) Inventors: Papapida PORNSURIYASAK, Chatuchak Bangkok (TH); Sophon KAEOTHIP, Chatuchak Bangkok (TH)

(73) Assignee: PTT Global Chemical Public Company Limited, Chatuchak, Bangkok (TH)

(21) Appl. No.: 16/618,902

(22) PCT Filed: May 30, 2018
(86) PCT No.: PCT/TH2018/000027

§ 371 Date: Dec. 3, 2019

(30) Foreign Application Priority Data
Jun. 5, 2017 (TH) 1701003120

(51) Int. Cl. C07C 233/49; C07C 233/47; C07C 233/83; C07C 231/14

(52) U.S. Cl. CPC C07C 233/49; C07C 231/14; C07C 233/83; C07C 233/47

(57) ABSTRACT

The present invention relates to a new diethanolamine derivative chelating agent having a high water solubility, a good chelating property, and biological degradation. The said new chelating agent can be prepared from reaction of diethanolamine and cyclic anhydride compound using lewis acid as the catalyst. The said process is uncomplicated, and does not use a severe condition, and also reduces the use of harmful chemicals.

### TECHNICAL FIELD

Chemistry relates to the biodegradable diethanolamine derivative chelating agent.

### BACKGROUND OF THE INVENTION

The chelating agent is a substance being used in the bonding with metal ions to separate ions of metals. At present, there are several groups of the chelating agent. The important factors to the effectiveness of the chelating agent to metal ions are molecular structure of the chelating agent and their function on the molecules. The main groups of the chelating agent used widely are aminopolycarboxylates and organophosphonates because of their excellent chelating property to metal ions e,g, ethylenediamineteraacetic acid (EDTA), nitrilotriacetic acid (NTA), diethylenetriaminepentaacetic acid (DTPA), diethylenetriamine penta methylene phosphonic acid (DTPMP) group, hydroxyethylidenediphosphonic acid (HEDP), and nitrilotrimethylenephosphonic acid (NTMP).

However, the said chelating agent groups have a limitation in their natural degradable and high toxicity. Therefore, there have been a development of chelating agent having a good metal chelating property and natural degradable in order to reduce their environmental effects.

The examples of degradable amino polycarbonate chelating agents are methylglycinediacetic acid (MGDA), polyaspartic acid (DS), glutamic acid N,N-diacetic acid (GLDA), iminodisuccinic acid (IDS), dihydroxyethylglycine (DHEG), and dihydroxyethylaspartate (DHEA). Although, the said natural degradable chelating agents are made industrially, they are not popular in the market because of their low to medium chelating property. Moreover, their precursors such as glutamic acid, aspartic acid are expensive and their reactants are highly toxic and not environmental friendly.

Generally, several methods are used with the synthetic organic chemicals to be bonded with carbon-carbon atoms and carbon atom to heteroatoms. In recent years, Michael reaction and hetero-Michael addition reaction are widely used to compare with Mannich or Aldol reactions because both Mannich and Aldol reactions require severe reaction conditions, long reaction times, and highly use of strong basic reactions. On the other hand, the hetero-Michael addition reaction provides a good reaction even under conditions of low amount of strong basic catalyst or lewis acid. Therefore, there have been research and development to synthesis organic substance by Michael reaction and hetero-Michael addition reaction to reduce the use of highly toxic reactants and non-environmental friendly reactions.

The preparation process of derivatives using diethanolamine as precursor with Michael reaction and hetero-Michael addition reaction has been disclosed in several patent documents as following.

U.S. Pat. No. 6,504,054B1 discloses the synthesis of aspartic acid derivatives chelating agent using L-aspartic ethoxylate or diethanolamine as precursor followed by an addition reaction using lanthanum as catalyst. U.S. Pat. No. 6,590,120B1 discloses the synthesis of diethanolamine derivative using alkaline metal salt or alkaline earth metal salt of maleic acid for an addition reaction on diethanolamine having substituent on nitrogen position. The catalysts used in this reaction are lanthanide metal or alkaline earth metal. The addition reaction using lanthanide metal as catalyst provides addition of maleate at nitrogen and oxygen positions of diethanolamine. Similarly, US20130204035 discloses the synthesis of a mixture of aspartic acid derivatives and aspartic diethoxy succinate acid by addition reaction of diethanolamine and maleate salt under basic condition using lanthanoid catalyst. However, the use of transition metal catalyst is costly and requires the purification step to separate catalyst from the desired product.

JPH08208569 discloses the diethanolamine derivative chelating agent using reaction of diethanolamine and maleic acid or maleic acid salt via addition reaction using co-catalysts of sodium hydroxide and calcium hydroxide. However, this process required a separation step of catalyst causing a difficulty to the synthesis process.

Moreover, the process for preparing diethanolamine derivative via addition reaction using acid catalyst was disclosed. For example, KR20150028464 discloses the synthesis of diethanolamine derivatives as photocurable resin composition using acetic acid as solvent and catalyst. However, the said process requires high amount of strong acid, and severe reaction conditions which might harm the reactor.

CN101921206 discloses the synthesis of diethanolamine derivative via addition reaction of diethanolamine and succinic anhydride using dimethyl formamide as solvent, providing N,N-di-monoethyl succin-4-amide−1-butyric acid as product to be used as plasticizer in melt mixing process in the production of polybutylene succinate (PBS). Although, the said process has several advantages such as broad range reaction temperature and reusable of dimethyl formamide solvent, dimethyl formamide has high boiling point which requires high energy to distill the solvent to be reusable.

In some case, the Michael reaction and hetero-Michael addition reaction can be reacted well under a condition with lewis acid catalyst such as bismuth(III) nitrate (Bi(NO₃)₃), palladium(II) acetate (Pd(OAc)₂), bis(1,5-cyclooctadiene)rhodium(I) tetrafluoroborate ([Rh(COD)₂] BF₄), indium(III) chloride (InCl₃), chloro(triphenylphosphine)gold(I)/silver trifluoromethanesulfonate (Ph₃PAuCl/AgOTf), or lanthanum(III) trifluoromethanesulfonate (La(OTf)₃) including the use of special process such as ultrasound microwave, high pressure reaction, or ionic liquid. However, the foregoing process uses transition metal catalyst which is difficult to be removed in the final step and it is an expensive process. Therefore, the development of effective process, cheap, no contamination of heavy metal, with low amount of catalyst to avoid side reaction such as polymerization reaction of precursor during reactions is very necessary.

The present invention aims to prepare the diethanolamine derivative having high water solubility, good chelating property, and biodegradable, wherein said process is uncomplicated, and does not use a severe condition, and also reduces the use of harmful chemicals.

### SUMMARY OF THE INVENTION

The present invention aims to propose the new chelating agent synthesized from diethanolamine having high water solubility, good chelating property, and biodegradable. The structure of the said new chelating agent is shown as (I)

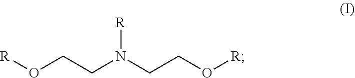

wherein R selected from groups with structure

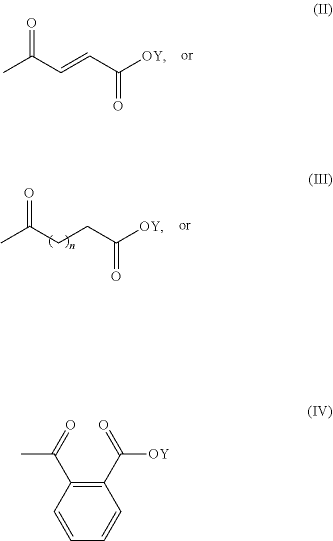

when n is integer from 1 to 2, and y represents a hydrogen atom, an alkaline metal atom, or an alkaline earth metal atom.

In another of the aspect of the present invention is to propose a process for preparing the new chelating agent, wherein said process is uncomplicated, and does not use a severe condition, and also reduces the use of harmful chemicals.

### BRIEF DESCRIPTION OF THE DRAWINGS

FIG. 1 shows the biodegradable property of chelating ligand 1.

FIG. 2 shows the biodegradable property of chelating ligand 2.

### DESCRIPTION OF THE INVENTION

The present invention relates to a new diethanolamine derivative chelating agent, wherein the said new chelating agent has high water solubility, good chelating property, and biologically degradable as will be described according to the following embodiments.

Any aspect showed herein is meant to include its application to other aspects of this invention unless stated otherwise.

### Definitions

Technical terms or scientific terms used here have definitions as by an ordinary person skilled in the art unless stated otherwise.

Any tools, equipment, methods, or chemicals named here mean tools, equipment, methods, or chemicals being used commonly by an ordinary person skilled in the art unless stated otherwise that they are tools, equipment, methods, or chemicals specific only in this invention.

Use of singular noun or singular pronoun with “comprising” in claims or specification means “one” and including “one or more”, “at least one”, and “one or more than one”.

All compositions and/or methods disclosed and claims in this application aim to cover embodiments from any action, performance, modification, or adjustment without any experiment that significantly different from this invention, and obtaining the object with utility and resulting the same as the present embodiment according to an ordinary person ordinary skilled in the art without specifically stated in claims. Therefore, substitutable or similar object to the present embodiment, including any little modification or adjustment that clearly seen by an ordinary person skilled in the art should be construed as remains in spirit, scope, and concept of invention as appeared in appended claims.

Throughout this application, term “about” means any number that appeared or showed herein that could be varied or deviated from any error of equipment, method, or personal using said equipment or method.

“Chelating agent” means including organic substance that can chelate the positive charge elements such as iron, zinc, copper, cobalt, and manganese, wherein the chelating agent is surround positive charge ions of the metal elements obtaining the complex compound with metal chelated in the molecule so that the negative charge from outside cannot react to. This combining reaction is called chelation.

“Degradable chelating agent” means including the biodegradable chelating agent such as degradable by heat, sunlight, or microorganisms.

“Alkaline metal or alkaline earth metal groups” means including group 1 or 2 elements in the element table, wherein group 1 elements or alkaline metal elements are lithium, sodium, potassium, rubidium, cesium, and francium, and group 2 elements or alkaline earth metal elements are beryllium, magnesium, calcium, strontium, barium, and radium.

“Lewis acid” is molecule or ion that can accept electron pair from other ions or molecules by coordinate bonding.

Hereafter, the invention embodiments are shown without any purpose to limit any scope of the invention.

The present invention relates to the new diethanolamine derivative chelating agent, wherein the said diethanolamine derivative chelating agent is shown according to structure (I)

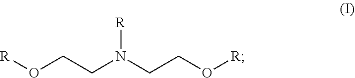

wherein:

R selected from groups with structure

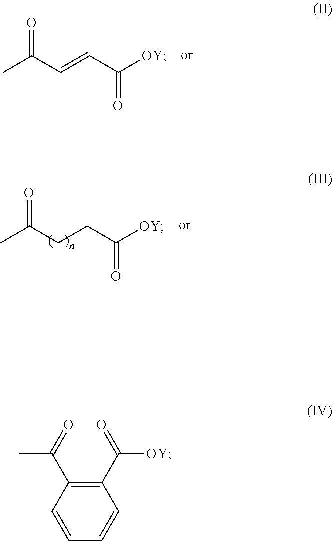

when n is integer from 1 to 2, and y represents a hydrogen atom, an alkaline metal atom, or an alkaline earth metal atom.

In one embodiment, the structure of the diethanolamine derivative chelating agent is

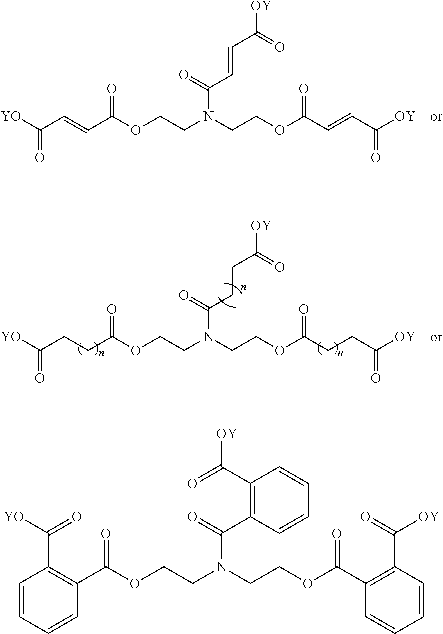

when n is integer from 1 to 2, preferably, n=1, and y represents a hydrogen atom, an alkaline metal atom, or an alkaline earth metal atom.

The another objective of the invention is to propose the process for preparing the new chelating agent, wherein the said process is the reaction of diethanolamine and cyclic anhydride compound, using lewis acid as the catalyst. The said process is uncomplicated, and does not use a severe condition, and also reduces the use of harmful chemicals.

In one embodiment, the process for preparing the diethanolamine derivatives chelating agent comprises the following steps;

a) mixing diethanolamine and cyclic anhydride compound in equivalent mole ratio from 1:1 to 1:5 in organic solvent, preferably 1:2 to 1:5; and

b) adding lewis acid catalyst into mixture obtained from a).

In one embodiment, in step a) the organic solvent selected from 1,4-dioxane, 1,2-dichloroethane, dichloromethane, or mixture thereof. Preferably, the organic solvent for step a) is dichloromethane.

In one embodiment, the cyclic anhydride compound in step a) selected from maleic anhydride, succinic anhydride, glutaric anhydride, or phthalic anhydride. Preferably, the cyclic anhydride compound is selected from maleic anhydride, succinic anhydride, or phthalic anhydride. More preferably, the cyclic anhydride compound is selected from maleic anhydride or succinic anhydride. Most preferably, the cyclic anhydride compound is maleic anhydride.

In one embodiment, the lewis acid catalyst in step b) selected from boron trifluoride (BF₃) in organic solvent, zinc chloride (ZnCl₂), aluminium chloride (AlCl₃), tin chloride (SnCl₂), or mixture thereof.

Preferably, the lewis acid catalyst is boron trifluoride diethyl ethylate (BF₃OEt₂).

In one embodiment, the process for preparing the diethanolamine derivatives chelating agent according to the present invention, wherein the said process is operated at the temperature from ambient temperature to 80° C. Preferably, the said process is operated at the temperature from 40° C. to 60° C.

The said process for preparing the diethanolamine derivatives chelating agent may comprise the purification step, wherein the purification method selected from, but not limited to solvent extraction and complete crystallization methods.

In another embodiment, the diethanolamine derivatives chelating agent according to the invention can be used to chelate metal ions, but not limited to aluminium (Al³⁺), barium (Ba²⁺), calcium (Ca²⁺), cadmium (Cd²⁺), cobolt (Co²⁺), copper (Cu²⁺), iron (II) (Fe²⁺), iron (III) (Fe³⁺), mercury (Hg²⁺), magnesium (Mg²⁺), manganese (Mn²⁺), nickel (Ni²⁺), tin (Pb²⁺), strontium (Sr²⁺), or zinc (Zn²⁺).

The following parts aim for describing the embodiments of the invention only, not for limiting the scope of this invention in any way.

### Example 1: The Preparation of the Diethanolamine Derivative from Reaction with Maleic Anhydride (Ligand 1)

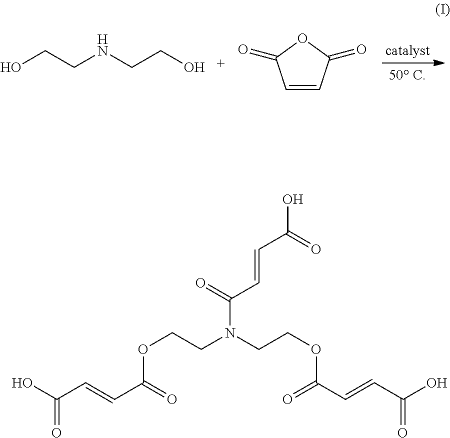 [SMILES: OCCNCCO ; O=C1C=CC(=O)O1 ; O=C(O)/C=C/C(=O)OCCN(CCOC(=O)/C=C/C(=O)O)C(=O)/C=C/C(=O)O]

The diethanolamine derivatives and maleic anhydride can be synthesized by the reaction according to equation (I). The diethanolamine precursor, maleic anhydride, and selected catalyst were added into about 15-20 mL of dichloromethane solvent, wherein the amounts of each compounds were shown in table 1. Then, the obtained mixture was refluxed at the temperature of 50° C. until the reaction was completed. The amount of precursors being used was monitored. The solvent was removed from the obtained product. The obtained product was dissolved in 50 mL of distilled water and washed with dichloromethane for at least 3 times (30 mL each). After being washed, the obtained product was dried under vacuum and analyzed for its property using ¹H NMR spectroscopy and liquid chromatography mass spectrometry (LC/MS) technique. The % yield and calcium chelation value of the synthesized diethanolamine derivatives and maleic anhydride are shown in table 2.

Table 2 shows that the ratio of used precursors and type of catalysts affect the synthesis of ligand 1 product. When boron trifluoride diethyl ethylate was used as the catalyst (ligand 1-8 to ligand 1-11), the said reaction could provide good diethanolamine derivatives and maleic anhydride, which also provide good chelation to the metal ions.

TABLE 1
The amount of precursors and reaction time in the synthesis
reaction of diethanolamine derivatives and maleic anhydride

| | | maleic | | |
|---|---|---|---|---|
| | diethanolamine | anhydride | | reaction |
| | amount | amount | | time |
| | (mmole) | (mmole) | type of catalyst* | (hour) |
| ligand 1-1 | 19 | 57 | sulfuric acid | 4 |
| | | | (H₂SO₄) | |
| ligand 1-2 | 19 | 57 | zinc chloride | 24 |
| | | | (ZnCl₂) | |
| ligand 1-3 | 19 | 57 | aluminium chloride | 24 |
| | | | (AlCl₃) | |
| ligand 1-4 | 19 | 57 | tin chloride | 5 |
| | | | (SnCl₂) | |
| ligand 1-5 | 19 | 57 | formic acid | 6 |
| | | | (HCOOH) | |
| ligand 1-6 | 19 | 57 | para-toluence | 4 |
| | | | sulfonic acid | |
| | | | (TsOH) | |
| ligand 1-7 | 19 | 38 | boron trifluoride | 5 |
| | | | diethyl ethylate | |
| | | | (BF₃OEt₂) | |
| ligand 1-8 | 19 | 57 | boron trifluoride | 5 |
| | | | diethyl ethylate | |
| | | | (BF₃OEt₂) | |
| ligand 1-9 | 19 | 95 | boron trifluoride | 5 |
| | | | diethyl ethylate | |
| | | | (BF₃OEt₂) | |
| ligand 1-10 | 19 | 57 | boron trifluoride | 12 |
| | | | diethyl ethylate | |
| | | | (BF₃OEt₂) | |
| ligand 1-11 | 19 | 57 | boron trifluoride | 24 |
| | | | diethyl ethylate | |
| | | | (BF₃OEt₂) | |

*the amount of catalyst in every test was 1.9 mmole

TABLE 2
The % yield and calcium chelation value of the
diethanolamine derivatives and maleic anhydride

| | % yield | calcium chelation value * |
|---|---|---|
| ligand 1-1 | 26 | 4 |
| ligand 1-2 | 37 | 15 |
| ligand 1-3 | 11 | 13 |
| ligand 1-4 | 31 | 9 |
| ligand 1-5 | 15 | 3 |
| ligand 1-6 | 0 | 0 |
| ligand 1-7 | 72 | 46 |
| ligand 1-8 | 89 | 58 |
| ligand 1-9 | 85 | 83 |
| ligand 1-10 | 90 | 62 |
| ligand 1-11 | 93 | 60 |

* the calcium chelation value was obtained from crude of ligand 1-1 to ligand 1-11 without purification

### Example 2: The Preparation of the Diethanolamine Derivative from Reaction with Succinic Anhydride (Ligand 2)

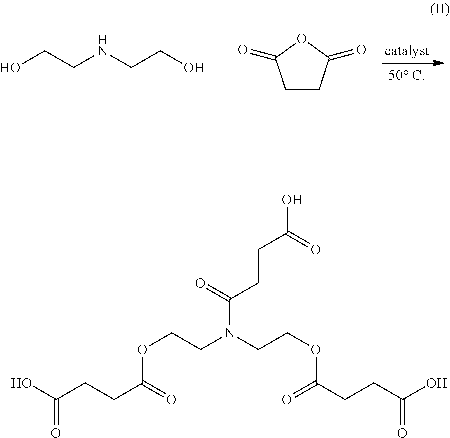 [SMILES: OCCNCCO ; O=C1CCC(=O)O1 ; O=C(O)CCC(=O)OCCN(CCOC(=O)CCC(=O)O)C(=O)CCC(=O)O]

The diethanolamine derivatives and succinic anhydride can be synthesized by the reaction according to equation (II). The diethanolamine precursor, succinic anhydride, and selected catalyst were added into about 15-20 mL of dichloromethane solvent, wherein the amounts of each compounds were shown in table 3. Then, the obtained mixture was refluxed at the temperature of 50° C. until the reaction was completed. The amount of precursors being used was monitored. The solvent was removed from the obtained product. The obtained product was dissolved in distilled water (50 mL) and washed with dichloromethane for at least 3 times (30 mL each). After being washed, the obtained product was dried under vacuum and analyzed for its property using ¹H NMR spectroscopy and liquid chromatography mass spectrometry (LC/MS) technique. The % yield and calcium chelation value of the synthesized diethanolamine derivatives and maleic anhydride are shown in table 4.

Table 4 shows the ratio of used precursors and type of catalysts affect the synthesis of ligand 2 product. When boron trifluoride diethyl ethylate was used as the catalyst (ligand 2-5 to ligand 2-9), the said reaction could provide good diethanolamine derivatives and succinic anhydride, which also provide good chelation to the metal ions.

TABLE 3
The amount of precursors and reaction time in the synthesis
reaction of diethanolamine derivatives and succinic anhydride

| | | succinic | | |
|---|---|---|---|---|
| | diethanolamine | anhydride | | reaction |
| | amount | amount | | time |
| | (mmole) | (mmole) | type of catalyst* | (hour) |
| ligand 2-1 | 19 | 57 | sulfuric acid | 7 |
| | | | (H₂SO₄) | |
| ligand 2-2 | 19 | 57 | zinc chloride | 24 |
| | | | (ZnCl₂) | |
| ligand 2-3 | 19 | 57 | aluminium chloride | 6 |
| | | | (AlCl₃) | |
| ligand 2-4 | 19 | 57 | tin chloride | 25 |
| | | | (SnCl₂) | |
| ligand 2-5 | 19 | 38 | boron trifluoride | 5 |
| | | | diethyl ethylate | |
| | | | (BF₃OEt₂) | |
| ligand 2-6 | 19 | 57 | boron trifluoride | 5 |
| | | | diethyl ethylate | |
| | | | (BF₃OEt₂) | |
| ligand 2-7 | 19 | 95 | boron trifluoride | 5 |
| | | | diethyl ethylate | |
| | | | (BF₃OEt₂) | |
| ligand 2-8 | 19 | 57 | boron trifluoride | 12 |
| | | | diethyl ethylate | |
| | | | (BF₃OEt₂) | |
| ligand 2-9 | 19 | 57 | boron trifluoride | 24 |
| | | | diethyl ethylate | |
| | | | (BF₃OEt₂) | |

*the amount of catalyst in every test was 1.9 mmole

TABLE 4
The % yield and calcium chelation value of the diethanolamine
derivatives and succinic anhydride

| | % yield | calcium chelation value * |
|---|---|---|
| ligand 2-1 | 15 | 5 |
| ligand 2-2 | 17 | 8 |
| ligand 2-3 | 68 | 55 |
| ligand 2-4 | 35 | 21 |
| ligand 2-5 | 81 | 45 |
| ligand 2-6 | 94 | 87 |
| ligand 2-7 | 91 | 42 |
| ligand 2-8 | 89 | 83 |
| ligand 2-9 | 90 | 85 |

* the calcium chelaton value was obtained from crude of ligand 2-1 to ligand 2-9 without purification

### Example 3: The Preparation of the Diethanolamine Derivatives from Reaction with Phthalic Anhydride (Ligand 3)

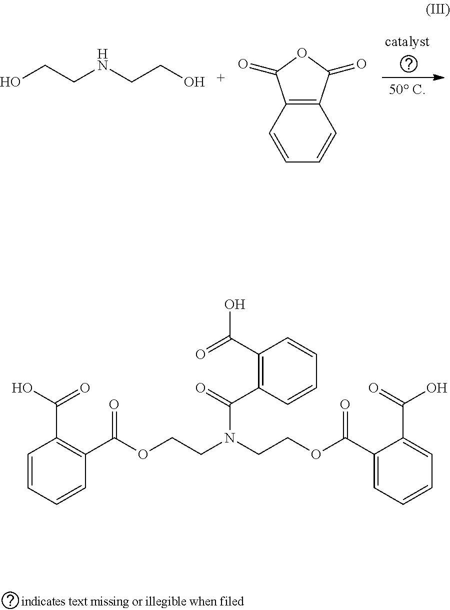 [SMILES: OCCNCCO ; O=C1OC(=O)c2ccccc21 ; O=C(O)c1ccccc1C(=O)OCCN(CCOC(=O)c1ccccc1C(=O)O)C(=O)c1ccccc1C(=O)O]

The diethanolamine derivatives and succinic anhydride can be synthesized by the reaction according to equation (III). The diethanolamine precursor, phthalic anhydride, and selected catalyst were added into about 15-20 mL of dichloromethane solvent, wherein the amounts of each compounds were shown in table 5. Then, the obtained mixture was refluxed at the temperature of 50° C. until the reaction was completed. The amount of precursors being used was monitored. The solvent was removed from the obtained product. The obtained product was dissolved in distilled water (50 mL) and washed with dichloromethane for at least 3 times (30 mL each). After being washed, the obtained product was dried under vacuum and analyzed for its property using ¹H NMR spectroscopy and liquid chromatography mass spectrometry (LC/MS) technique. The % yield and calcium chelation value of the synthesized diethanolamine derivative and phthalic anhydride are shown in table 6.

Table 6 shows the ratio of used precursors and type of catalysts affect the synthesis of ligand 3 product. When boron trifluoride diethyl ethylate was used as the catalyst (ligand 3-5 to ligand 3-7), the said reaction could provide good diethanolamine derivatives and phthalic anhydride, which also provide good chelation to the metal ions.

TABLE 5
The amount of precursors and reaction time in the synthesis
reaction or diethanolamine derivatives and phthalic anhydride

| | | phthalic | | |
|---|---|---|---|---|
| | diethanolamine | anhydride | | reaction |
| | amount | amount | | time |
| | (mmole) | (mmole) | type of catalyst* | (hour) |
| ligand 3-1 | 19 | 57 | sulfuric acid | 12 |
| | | | (H₂SO₄) | |
| ligand 3-2 | 19 | 57 | zinc chloride | 24 |
| | | | (ZnCl₂) | |
| ligand 3-3 | 19 | 57 | aluminium chloride | 15 |
| | | | (AlCl₃) | |
| ligand 3-4 | 19 | 57 | tin chloride | 16 |
| | | | (SnCl₂) | |
| ligand 3-5 | 19 | 38 | boron trifluoride | 3 |
| | | | diethyl ethylate | |
| | | | (BF₃OEt₂) | |
| ligand 3-6 | 19 | 57 | boron trifluoride | 3 |
| | | | diethyl ethylate | |
| | | | (BF₃OEt₂) | |
| ligand 3-7 | 19 | 95 | boron trifluoride | 3 |
| | | | diethyl ethylate | |
| | | | (BF₃OEt₂) | |

*the amount of catalyst in every test was 1.9 mmole

TABLE 6
The % yield and calcium chelation value of the diethanolamine
derivatives and phthalic anhydride

| | % yield | calcium chelation value * |
|---|---|---|
| ligand 3-1 | 22 | 25 |
| ligand 3-2 | 18 | 15 |
| ligand 3-3 | 66 | 81 |
| ligand 3-4 | 54 | 70 |
| ligand 3-5 | 61 | 75 |
| ligand 3-6 | 75 | 88 |
| ligand 3-7 | 65 | 76 |

* the calcium, chelation value was obtained from crude of ligand 3-1 to ligand 3-7 without purification

### Example 4: The Analysis of Stability Constant of the Synthesized Sheltie Agent and Metal Ions

The analysis of stability constant between the synthesized chelating agent and metal ions can be performed by complex titration wherein the stability constant of the synthesized chelating agent and metal ions comparing to the previous disclosed degradable chelating agent can be performed according to table 7.

From table 7, it is found that when comparing to the previous disclosed degradable chelating agent, the new chelating agent according to the invention can chelate many metal ions with good ability similar to the previous disclosed degradable chelating agent.

TABLE 7
The stability constant of the synthesized chelating agent and metal ions comparing to the
previous disclosed degradable chelating agent

| | | | | | | | | N-Bis[2-(1,2- | | |
|---|---|---|---|---|---|---|---|---|---|---|
| | | | glutamic | | | ethylene | | dicarboxy | | |
| | Ethylene | nitrile | acid N,N- | | | diamine- | | ethoxy) | | |
| | diaminetetra | triacetic | diacetic | methyl glycine | Imino | N,N- | ethanol | ethyl aspartic | | |
| | acetic acid | acid | acid | diacetic acid | disuccinic acid | disuccinic acid | diglycine | acid | | |
| metal ions | (EDTA) | (NTA) | (GLDA) | (MGDA) | (IDS) | (EDDS) | (EDG) | (BCA6) | ligand 1 | ligand 2 |
| aluminium | 16.4 | 11.4 | 12.2 | — | 14.1 | — | 7.7 | 6.14 | 10.7 | 11.4 |
| (Al³⁺⁾ | | | | | | | | | | |
| barium | 7.9 | 4.8 | 3.5 | 4.9 | 2.1 | 3.0 | 3.4 | 6.1 | 7.8 | 6.2 |
| (Ba²⁺) | | | | | | | | | | |
| calcium | 10.7 | 6.4 | 5.9 | 7.0 | 5.2 | 4.6 | 4.7 | 7.71 | 9.3 | 10.1 |
| (Ca²⁺) | | | | | | | | | | |
| cadmium | 16.5 | 9.8 | 9.1 | — | 8.4 | 16.4 | 7.4 | 11.09 | 8.5 | 7.2 |
| (Cd²⁺) | | | | | | | | | | |
| cobolt | 16.5 | 10.4 | 10.0 | — | 10.5 | 13.6 | 8.0 | — | 10.4 | 7.5 |
| (Co²⁺) | | | | | | | | | | |
| copper | 18.8 | 13.0 | 13.1 | 13.9 | 13.1 | 18.4 | 11.8 | 13.08 | 12.8 | 10.5 |
| (Cu2+) | | | | | | | | | | |
| iron | 14.3 | 8.9 | 8.7 | 8.1 | 8.2 | — | 13.4 | — | 14.8 | 12.3 |
| (Fe²⁺) | | | | | | | | | | |
| iron | 25.1 | 15.9 | 11.7 | 16.5 | 15.2 | 22.0 | 21.6 | 17.3 | 9.5 | 7.5 |
| (Fe³⁺) | | | | | | | | | | |
| mercury | 21.5 | 14.3 | 14.3 | — | 14.9 | — | 19.8 | 14.85 | 7.8 | 9.5 |
| (Hg²⁺) | | | | | | | | | | |
| magnesium | 8.8 | 5.5 | 5.2 | 5.8 | 6.1 | 6.0 | 6.2 | 5.98 | 8.3 | 8.1 |
| (Mg²⁺) | | | | | | | | | | |
| manganese | 13.9 | 7.5 | 7.6 | 8.4 | 7.7 | — | 10.0 | 9.28 | 10.4 | 9.1 |
| (Mn²⁺) | | | | | | | | | | |
| nickel | 18.4 | 11.5 | 10.9 | 12.0 | 12.2 | 16.7 | 18.2 | — | 5.0 | 5.4 |
| (Ni²⁺) | | | | | | | | | | |
| tin | 18.0 | 11.5 | 10.5 | — | 11.0 | 12.7 | 13.6 | 10.82 | 11.4 | 9.9 |
| (Pb²⁺) | | | | | | | | | | |
| strontium | 8.7 | 5.0 | 4.1 | — | — | — | 5.2 | — | 7.7 | 5.3 |
| (Sr²⁺) | | | | | | | | | | |
| zinc | 16.5 | 10.7 | 10.0 | 10.9 | 10.8 | 13.4 | 15.2 | 11.34 | 9.0 | 7.9 |
| (Zn²⁺) | | | | | | | | | | |

### Example 4: The Test of Biodegradation of the Synthesized Chelating Agent

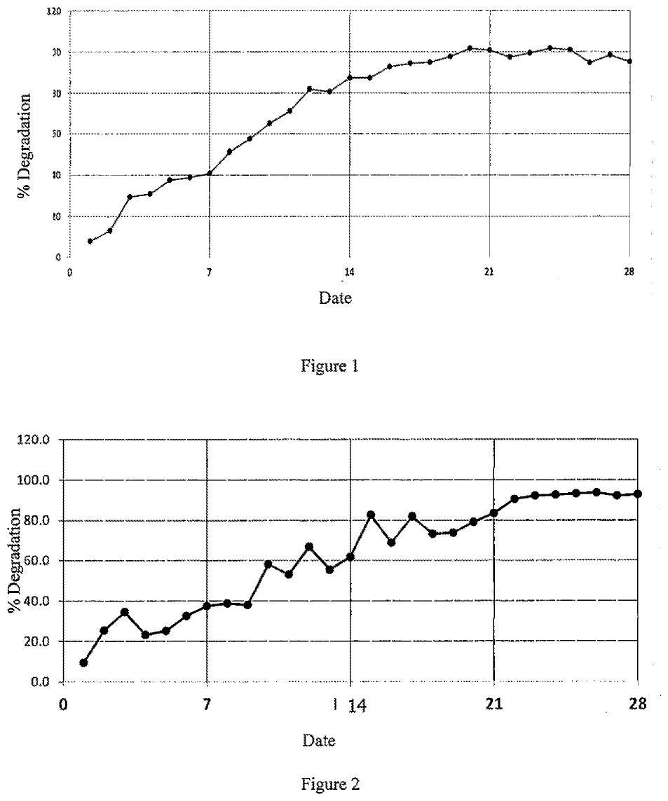

The test of biodegradation of the synthesized chelating agent can be performed using OECD 301D closed bottle test by determining the decomposition degree (Dt), the biochemical oxygen demand (BOD), the CO₂ emission within 28 days, wherein the biodegradation of the synthesized chelating agent (ligand 1 and 2) are shown in FIGS. 1 and 2.

### PREFERRED EMBODIMENT OF THE INVENTION

Preferred embodiment of the invention is as provided in the description of the invention.

1. A diethanolamine derivative chelating agent according to structure (I) 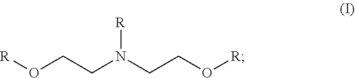
wherein:
R can be selected from groups with structure 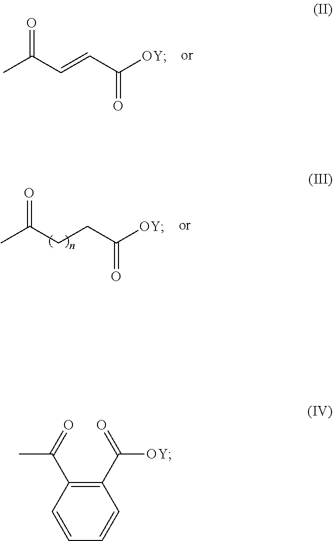
when n is integer from 1 to 2, and y represents a hydrogen atom, an alkaline metal atom, or an alkaline earth metal atom.

2. The diethanolamine derivative chelating agent according to claim 1, wherein the structure of the diethanolamine derivative chelating agent is 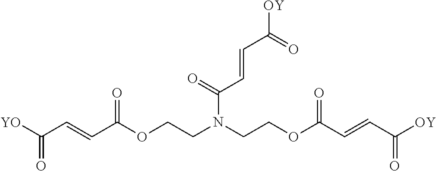
when y represents a hydrogen atom, an alkaline metal atom, or an alkaline earth metal atom.

3. The diethanolamine derivative chelating agent according to claim 1, wherein the structure of the diethanolamine derivative chelating agent is 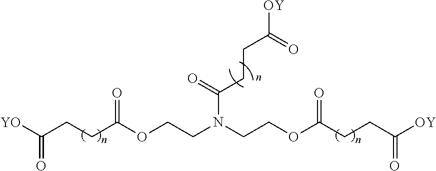
when n is integer from 1 to 2, and y represents a hydrogen atom, an alkaline metal atom, or an alkaline earth metal atom.

4. The diethanolamine derivative chelating agent according to claim 3, wherein n is 1.

5. The diethanolamine derivative chelating agent according to claim 1, wherein the structure of the diethanolamine derivative chelating agent is 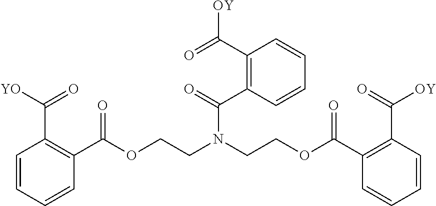
when y represents a hydrogen atom, an alkaline metal atom, or an alkaline earth metal atom.

6. A process for preparing the diethanolamine derivative chelating agent according to claim 1, wherein said process comprises the following steps:
a) mixing diethanolamine and cyclic anhydride compound in equivalent mole ratio from 1:1 to 1:5 in organic solvent; and
b) adding lewis acid catalyst into mixture obtained from a).

7. The process for preparing the diethanolamine derivative chelating agent according to claim 6, wherein the organic solvent in step a) can be selected from 1,4-dioxane, 1,2-dichloroethane, dichloromethane, or mixture thereof.

8. The process for preparing the diethanolamine derivative chelating agent according to claim 7, wherein the organic solvent in step a) is dichloromethane.

9. The process for preparing the diethanolamine derivative chelating agent according to claim 6, wherein the cyclic anhydride compound in step a) is selected from maleic anhydride, succinic anhydride, glutaric anhydride, or phthalic anhydride.

10. The process for preparing the diethanolamine derivative chelating agent according to claim 9, wherein the cyclic anhydride compound is selected from maleic anhydride, succinic anhydride, or phthalic anhydride.

11. The process for preparing the diethanolamine derivative chelating agent according to claim 6, wherein the equivalent mole ratio between diethanolamine and cyclic anhydride compound is from 1:2 to 1:5.

12. The process for preparing the diethanolamine derivative chelating agent according to claim 6, wherein the lewis acid catalyst in step b) is selected from boron trifluoride (BF₃) in organic solvent, zinc chloride (ZnCl₂), aluminium chloride (AlCl₃), tin chloride (SnCl₂), or mixture thereof.

13. The process for preparing the diethanolamine derivative chelating agent according to claim 12, wherein the lewis acid catalyst is selected from boron trifluoride in organic solvent.

14. The process for preparing the diethanolamine derivative chelating agent according to claim 13, wherein the lewis acid catalyst is selected from boron trifluoride in diethyl ether.

15. The process for preparing the diethanolamine derivative chelating agent according to claim 14, wherein the lewis acid catalyst is boron trifluoride diethyl ethylate (BF₃OEt₂).

16. The process for preparing the diethanolamine derivative chelating agent according to claim 6, wherein the said process is operated at the temperature from ambient temperature to 80° C.

17. The process for preparing the diethanolamine derivative chelating agent according to claim 16, wherein the said process is operated at the temperature from 40° C. to 60° C.
